(19) United States
(12) Patent Application Publication
(10) Pub. No.: US 2008/0033309 A1
(43) Pub. Date: Feb. 7, 2008

(54) Blood pressure measurement device storing measurement data

(75) Inventors: Yoshihiko Sano, Kyoto-shi (JP); Takefumi Nakanishi, Nagaokakyo-shi (JP); Takahide Tanaka, Otsu-shi (JP); Yukiya Sawanoi, Nara-shi (JP); Kenji Eda, Suita-shi (JP); Minoru Taniguchi, Uji-shi (JP); Hiroya Nakanishi, Kyoto-shi (JP); Tomonori Inoue, Kyoto-shi (JP); Tetsuya Nakada, Takatsuki-shi (JP)

Correspondence Address: MORRISON & FOERSTER LLP, 1650 TYSONS BOULEVARD, SUITE 400, MCLEAN, VA, 22102, US

(73) Assignee: OMRON HEALTHCARE Co., Ltd., Kyoto-shi (JP)

(21) Appl. No.: 11/812,382

(22) Filed: Jun. 18, 2007

Related U.S. Application Data
(63) Continuation of application No. 11/061,446, filed on Feb. 22, 2005.

(30) Foreign Application Priority Data
Feb. 25, 2004 (JP) 2004-049913

(51) Int. Cl. A61B 5/0225

(52) U.S. Cl. 600/490

(57) ABSTRACT

A blood pressure measurement device includes a fixed main body unit and a removable display unit. The main body unit includes a cuff mounted on a region for measurement of a living body, a control unit including a CPU for measuring a blood pressure with adjusting a pressure of the cuff, and a main body operation unit operated by a subject to externally provide instructions for a measurement operation of the control unit. The display unit includes a display memory storing data of the measurement with the control unit, a display portion displaying measurement data in the display memory, and a display operation unit operated by a subject to externally provide instructions for a display operation of the display portion. A measurement result stored in the display memory of the removable display unit can be displayed on the display portion by operating the display operation unit when the display unit is removed from the main body unit.

### BACKGROUND OF THE INVENTION

1. Field of the Invention

The present invention relates to a blood pressure measurement device and, more specifically, to a blood pressure measurement device storing measurement data in a removable portion mounted thereon.

2. Description of the Background Art

Conventionally, information associated with blood pressures measured at home and blood pressure measurement is sometimes recorded in a sphygmomanometer (a blood pressure gauge) for disclosure thereof in an external medical institution such as a hospital to receive treatment or guidance. As a method of disclosure of the information, a user writes on a sheet of paper the information associated with the blood pressure measurement recorded in the sphygmomanometer and then takes the paper, or takes a main body of the sphygmomanometer recording the information to the external medical institution such as a hospital to show a doctor the recorded information to receive treatment or guidance.

In the conventional method as such, however, incorrect information may be written by mistake, or complicated data processing may be subsequently required. In addition, the main body of the sphygmomanometer recording the information is heavy and bulky for carrying.

Therefore, a patent reference 1 (Japanese Patent Laying-Open No. 10-127587), for example, proposes a technique enabling information of blood pressure record to be read in another facility or with another system using a memory card. In addition, a patent reference 2 (Japanese Patent Laying-Open No. 2000-83912) proposes a sphygmomanometer including a sphygmomanometer main body unit and an arm band (a cuff) unit which can be used integrally or separately using a cable.

The sphygmomanometer having a removable memory card as disclosed in patent reference 1 is not practical because, to remove the memory card from the sphygmomanometer and read stored data with another system or the like, an additional mechanism for another system for accessing the memory card and displaying read data is required.

In addition, in a construction having the cuff unit and the main body unit separate from each other as disclosed in patent reference 2, the main body unit includes a display unit, an operation unit for the sphygmomanometer, and a control unit for controlling each portion of the sphygmomanometer which has a CPU (central processing unit) and a memory storing any kind of data including measurement data. For a use wherein the main body unit as such is carried to the aforementioned another system to display and ensure contents of the memory, the main body unit is heavy and bulky for carrying because the main body unit integrally has portions not needed for such use, such as the control unit for a whole sphygmomanometer and the operation unit for operating the sphygmomanometer.

### SUMMARY OF THE INVENTION

An object of the present invention is to provide a blood pressure measurement device having enhanced practical utility to display measurement data stored in a removable portion in a removed state of the portion.

A blood pressure measurement device according to an aspect of the present invention includes a main body unit fixed to the blood pressure measurement device and a display unit removably mounted on the blood pressure measurement device.

The main body unit includes a cuff mounted on a region for measurement of a living body, a control unit for measuring a blood pressure with adjusting a pressure of the cuff for blood pressure measurement, and a main body operation unit operated to externally provide instructions for a blood pressure measurement operation of the control unit. The cuff is integrally or separately attached to the main body unit.

The display unit includes a storage unit for storing data of blood pressure measured by the control unit, a display portion for displaying measurement data in the storage unit, and a display operation unit operated to externally provide instructions for a display operation of the display portion.

Therefore, since the measurement data in the storage unit can be displayed on the display portion by an operation of the display operation unit, the measurement data can be displayed and ensured without preparation of a specific display device when the display unit is removed and carried to a medical institution or the like for displaying.

In addition, since the display unit including the storage unit storing the measurement data of blood pressure measurement can be removed from the blood pressure measurement device, one can easily go to an external medical institution or the like carrying only the display unit which is smaller and lighter than the blood pressure measurement device.

Furthermore, as the main body operation unit for providing instructions for the blood pressure measurement operation and the display operation unit for providing instructions for the display operation are separately provided and only the display operation unit is provided on the display unit to be removed, reduction in size and weight of the display unit is enabled.

In addition, removing of the display unit during the blood pressure measurement is practical because the display unit can be moved to a certain position according to a posture for the measurement so that the display can be easily seen.

The display unit preferably further includes a display-side interface for communicating with an external portion and reads the measurement data in the storage unit for transmission to the external portion via the display-side interface. Therefore, the measurement data can be received and displayed with an external device of a transmitted side.

The display unit preferably updates the measurement data in the storage unit based on an instruction received from the external portion via the display-side interface.

Therefore, the measurement data stored in the storage unit of the display unit can be updated by transmission of an instruction from the external portion.

It is preferable that the instruction include additional data for storing in the storage unit. That is, the measurement data stored in the storage unit of the display unit can be updated with the additional data transmitted from the external portion.

It is preferable that the instruction instruct to delete the measurement data in the storage unit. That is, the measurement data stored in the storage unit of the display unit can be deleted with the instruction transmitted from the external portion.

The main body unit preferably further includes a main body-side interface for communicating with an external portion and reads the measurement data in the storage unit via the main body-side interface for transmission to the external portion.

Therefore, the measurement data in the storage unit of the display unit can be read from the main body unit and transmitted to an external device or the like.

The display unit preferably further includes a display-side interface for communicating with an external portion. The main body unit updates the measurement data in the storage unit via the main body-side interface and the display-side interface based on an instruction received from the external portion via the main body-side interface.

Therefore, the measurement data in the storage unit of the display unit can be updated based on the instruction received by the main body unit from the external portion.

Preferably, the display unit communicates with the main body unit via wire or wireless. Therefore, the display unit can communicate with the main body unit for transmitting or receiving measurement data or the like even when it is removed. In addition, use of the wireless increases flexibility in a location of the removed display unit because a run of a cable is not required.

The measurement data stored in the storage unit is preferably data corresponding to one or a plurality of blood pressure measurements.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a block diagram of a construction of a blood pressure measurement device according to a first embodiment.

FIGS. 2A-2C show examples of a manner of using the blood pressure measurement device according to the first embodiment.

FIGS. 3A and 3B show examples of display of measurement data.

FIG. 4 is a block diagram of a construction of a blood pressure measurement device according to a second embodiment.

FIGS. 5A and 5B show examples of a manner of using the blood pressure measurement device according to the second embodiment.

FIG. 6 shows an example of a manner of using a blood pressure measurement device according to a third embodiment.

### DESCRIPTION OF THE PREFERRED EMBODIMENTS

Each embodiment of the present invention will be described in the following referring to the drawings.

### First Embodiment

In a blood pressure measurement device according to a first embodiment, a memory storing information associated with blood pressure measurement, a display portion for displaying the information associated with blood pressure measurement, an operation unit for the blood pressure measurement device, and an operation unit for the display portion are provided integrally and can be removed from the blood pressure measurement device.

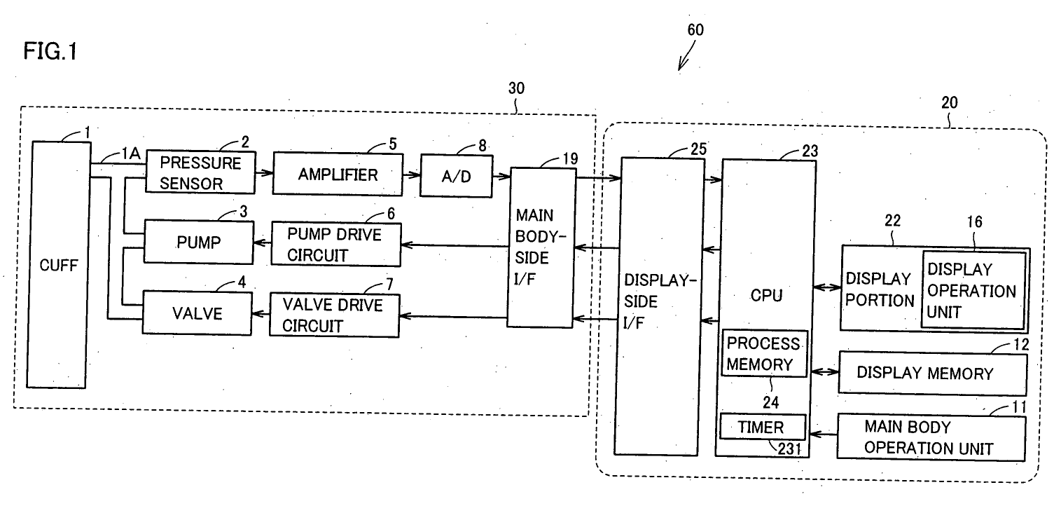

Referring to FIG. 1, a blood pressure measurement device 60 according to the first embodiment includes a main body unit 30 fixed to blood pressure measurement device 60 and an operation and display unit 20 removably provided on blood pressure measurement device 60. Main body unit 30 includes a cuff (an arm band) 1 for applying a pressure with an air pressure to a region for blood pressure measurement of a subject, a pressure sensor 2 for outputting a variation in a pulse pressure detected via cuff 1 as a signal of a pulse wave, an amplifier 5 amplifying and outputting a pulse wave signal output from pressure sensor 2, an A/D (analog/digital) converter 8 for converting the pulse wave signal output from amplifier 5 into a digital signal allowing data processing, a pump 3 and a valve 4 for adjusting a pressure (air pressure) level applied from cuff 1, a pump drive circuit 6 driving pump 3, a valve drive circuit 7 for adjusting opening and closing of valve 4, and a main body-side I/F (interface) 19 for communicating with an external portion. Cuff 1 is connected to pressure sensor 2, pump 3 and valve 4 via an air tube 1A.

Operation and display unit 20 includes a CPU (central processing unit) 23 for centralized control and monitoring of each portion of blood pressure measurement device 60, a display portion 22 for displaying various information regarding blood pressure measurement, a main body operation unit 11 for providing instructions for a measurement operation of blood pressure measurement device 60 from an external portion by a key operation or the like, a display memory 12 storing information for display on display portion 22, and a display-side I/F 25 for communicating with an external portion.

CPU 23 includes a timer 231 and a process memory 24 storing various programs and data for controlling operations such as the measurement operation of blood pressure measurement device 60 and a display operation via display portion 22. Timer 231 counts current time to output time information. Display portion 22 includes a display operation unit 16 having a key operated from an external portion for providing instructions for switching of displayed information or ON/OFF switching of the display. While operation and display unit 20 is mounted on blood pressure measurement device 60, main body-side I/F 19 and display-side I/F 25 are connected with each other. With this, data and signals can be transmitted and received between A/D converter 8, pump drive circuit 6 and valve drive circuit 7 on a side of main body unit 30 and CPU 23 on a side of operation and display unit 20.

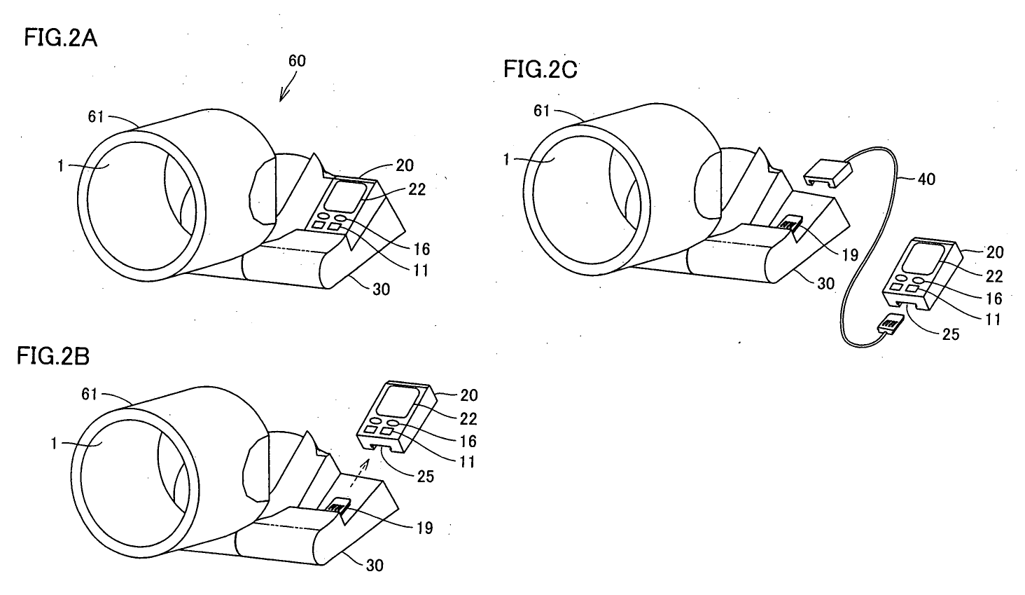

Each of FIGS. 2A, 2B and 2C shows a manner of using blood pressure measurement device 60 according to the first embodiment. FIG. 2A shows a state in which operation and display unit 20 is mounted on blood pressure measurement device 60 so that main body unit 30 and operation and display unit 20 are in an integral form. In the manner shown in FIG. 2A, when a subject passes an arm, a region for measurement, through a housing 61 including cuff 1 and operates main body operation unit 11 to instruct to start blood pressure measurement, CPU 23 starts processing for the blood pressure measurement based on a provided instruction and data specifying the subject from main body operation unit 11. The subject inputs the data specifying himself/herself via main body operation unit 11. During the measurement, a digital signal indicating data of a result of the measurement obtained via pressure sensor 2, amplifier 5 and A/D converter 8 is provided to CPU 23 via main body-side I/F 19 and display-side I/F 25. CPU 23 performs prescribed processing of blood pressure measurement data provided and writes the result to display memory 12 for storing and also to process memory 24 as required for storing. The measurement data stored in display memory 12 and process memory 24 includes blood pressure values, data specifying the subject and time information from timer 231. The time information indicate a measurement time. When the blood pressure measurement is ended, the subject removes the arm from housing 61.

After the blood pressure measurement, when the subject desires to show the result of the measurement in an external medical institution such as a hospital to receive a diagnosis, blood pressure measurement device 60 is used in a manner as shown in FIG. 2B. In FIG. 2B, the subject removes operation and display unit 20 from blood pressure measurement device 60 in a direction of an arrow indicated with a broken line. This brakes a connection between main body-side I/F 19 and display-side I/F 25.

As such, the subject can remove operation and display unit 20 from blood pressure measurement device 60 and carry operation and display unit 20 alone to the external medical institution such as a hospital. When display operation unit 16 of operation and display unit 20 is operated at the medical institution to instruct to display the result of the measurement, CPU 23 reads the measurement data previously stored in display memory 12 or process memory 24 responsive to this instruction, manipulates the data to obtain a format for displaying and displays the result via display portion 22.

Though the instruction regarding display for display portion 22 is provided to CPU 23 via display operation unit 16, the instruction may be provided by operation of main body operation unit 11. In addition, though the blood pressure measurement data is stored in display memory 12 or process memory 24, the data may be stored in only one of the memories.

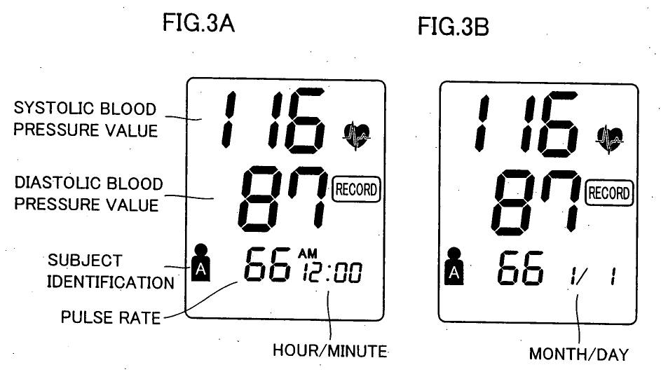

FIGS. 3A and 3B show examples of display of measurement data. In FIG. 3A, a display is provided for each subject, which display includes a measurement time (hour and minute), a result of the measurement, that is, a systolic blood pressure value, a diastolic blood pressure value and a pulse rate, and data specifying the subject. As shown in FIG. 3B, a measurement date may be indicated with month and day. In FIG. 3B, average values of systolic blood pressures, diastolic blood pressures and pulse rates on a certain measurement date and identification of the subject are displayed. One or a plurality of measurement data of a kind which can be displayed as shown in FIGS. 3A and 3B are associated with each other and stored in display memory 12 or process memory 24.

When operation and display unit 20 is removed from blood pressure measurement device 60 as shown in FIG. 2B, display-side I/F 25 and main body-side I/F 19 may be connected to each other with a cable 40 to enable communication therebetween, as shown in FIG. 2C. Though cable 40 connected between display-side I/F 25 and main body-side I/F 19 is described here, I/Fs other than main body-side I/F 19 and display-side I/F 25 may be provided on main body unit 30 and operation and display unit 20 for connecting cable 40.

In the manner shown in FIG. 2A, when the subject inserts a right arm into housing 61 from a front side of the drawing at a time of blood pressure measurement, a resulting posture makes it difficult for the subject to ensure displayed data on display portion 22 or to operate main body operation unit 11 or display operation unit 16. By connecting main body unit 30 with operation and display unit 20 via cable 40 as shown in FIG. 2C, operation and display unit 20 can be moved easily with connected cable 40 to a position which facilitates seeing of the display or operation by the subject, regardless of the posture for measurement.

It is to be noted that, though operation and display unit 20 and main body unit 30 are connected to each other with cable 40 to enable communication therebetween in FIG. 2C, they may be connected in a wireless manner with radio communication such as infrared communication.

In addition, though cuff 1 is integrally attached to main body unit 30 in this embodiment, it may be attached separately via air tube 1A.

### Second Embodiment

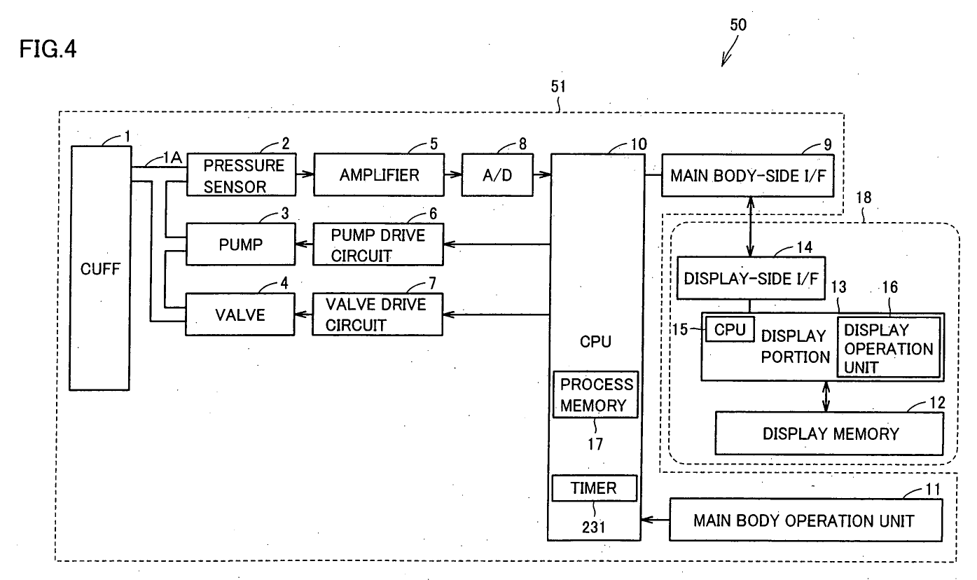

A second embodiment shows a blood pressure measurement device having a display unit for displaying blood pressure measurement data provided in a removable form. Referring to FIG. 4, a blood pressure measurement device 50 according to the second embodiment includes a main body unit 51 fixed to blood pressure measurement device 50 and a display unit 18 removably provided on blood pressure measurement device 50. Main body unit 51 includes cuff 1, pressure sensor 2, pump 3, valve 4, amplifier 5, pump drive circuit 6, valve drive circuit 7, A/D converter 8, a main body-side I/F 9 for communicating with an external portion, a CPU 10 including a process memory 17 and timer 231, and main body operation unit 11. Cuff 1 is connected to pressure sensor 2, pump 3 and valve 4 via air tube 1A. Process memory 17 stores programs and various data for CPU 10 to control various operations including a measurement operation. Main body operation unit 11 is externally operated to provide instructions for a blood pressure measurement operation.

Display unit 18 includes display memory 12 storing data for display including data of blood pressure measurement, a display portion 13 for displaying data stored in display memory 12, and a display-side I/F 14 for communicating with an external portion. Display portion 13 includes a CPU 15 for monitoring and controlling a display operation and display operation unit 16 externally operated to provide instructions for switching of displayed information or ON/OFF switching of the display. When display unit 18 is mounted on blood pressure measurement device 50, main body-side I/F 9 and display-side I/F 14 are connected to each other and data communication between CPU 15 and CPU 10 is enabled.

When display unit 18 is removed from blood pressure measurement device 50, main body-side I/F 9 and display-side I/F 14 may be connected to each other with a cable as shown in FIG. 2C, or they may be connected without a cable so as to allow wireless communication such as infrared communication therebetween.

In addition, though cuff 1 is integrally attached to main body unit 51 in this embodiment, it may be attached separately via air tube 1A.

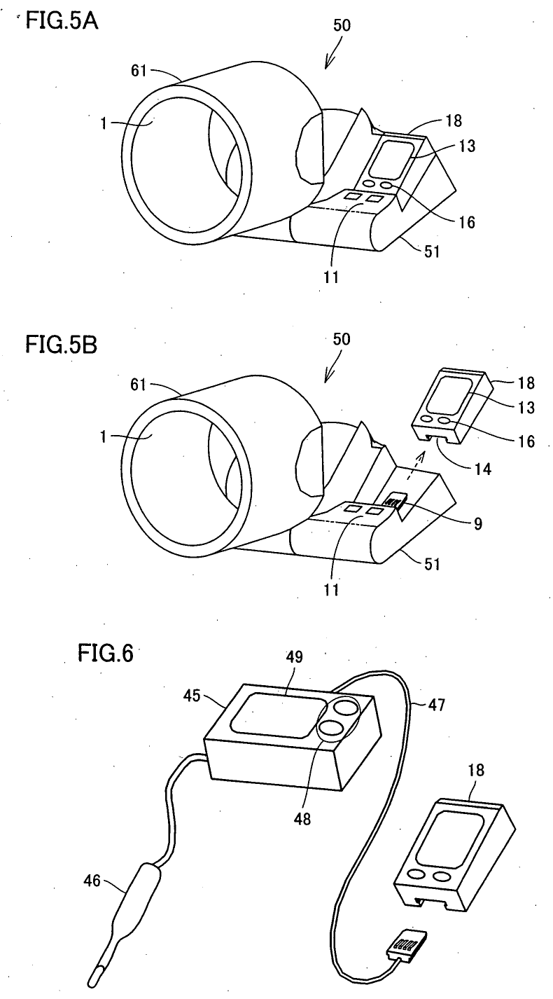

Each of FIGS. 5A and 5B shows a manner of using blood pressure measurement device 50 according to this embodiment. In the manner shown in FIG. 5A, display unit 18 is mounted on blood pressure measurement device 50 so that display unit 18 and main body unit 51 are in an integral form. For measuring a blood pressure in a state shown in FIG. 5A, a subject first inserts an arm as a region for measurement into housing 61. When it is ready for measurement, the subject operates main body operation unit 11 to instruct to start blood pressure measurement, and thus the blood pressure measurement is started. Measurement data is obtained as described in the first embodiment and provided to CPU 10. CPU 10 performs prescribed processing of the measurement data provided and stores the result in process memory 17 or display memory 12 of display unit 18.

When the measurement data is stored in display memory 12 in the state shown in FIG. 5A, CPU 10 outputs the measurement data to display unit 18 via main body-side I/F 9. In display unit 18, display-side I/F 14 inputs the measurement data from main body unit 51 for providing to display portion 13, and CPU 15 of display portion 13 stores the measurement data provided in display memory 12.

After the blood pressure measurement operation, when the subject shows the result of the measurement in an external medical institution such as a hospital to receive a diagnosis, treatment or the like, a manner as shown in FIG. 5B is employed.

In FIG. 5B, the subject removes display unit 18 from blood pressure measurement device 50 in a direction of an arrow indicated with a broken line, and carries the removed display unit 18 to the external medical institution such as a hospital. At the medical institution, display operation unit 16 of display unit 18 is operated to instruct to display the measurement data. When this instruction is input, CPU 15 reads the measurement data from display memory 12 and displays on display portion 13. Examples of the display in this situation are similar to those shown in FIGS. 3A and 3B described above.

According to the first and second embodiments, by removing operation and display unit 20 from blood pressure measurement device 60 or by removing display unit 18 from blood pressure measurement device 50, the subject can easily carry information regarding blood pressure measurement without being bulky, separately from a pressure level adjustment system for cuff 1 for the blood pressure measurement. Therefore, preparation of a specific separate display device is not required to display and ensure the information regarding blood pressure measurement on a place other than a place of the blood pressure measurement, for example, a place on which blood pressure measurement device 50 or 60 is located. It is practical because when the information is to be carried to an external institution for representation to a doctor to receive a diagnosis, for example, the doctor does not need to have a separate display device.

### Third Embodiment

Display memory 12 of display unit 18 described in the second embodiment can store additional information provided from a separate device other than blood pressure measurement device 50. Though a thermometer is applied as the separate device in a third embodiment, it is not limited to a thermometer and may be a device for measuring another kind of physiological information.

FIG. 6 shows an example of a manner of using the third embodiment. In FIG. 6, a thermometer main body 45 connecting a thermometer probe 46 to be pressed against a region for measurement of a subject to measure a body temperature and display unit 18 removed from blood pressure measurement device 50 are connected to each other via a cable 47 to enable communication therebetween. Display-side I/F 14 may be used as an interface of display unit 18 for connecting cable 47, or another interface may be provided. Thermometer main body 45 includes a microcomputer which is not shown, and the microcomputer performs prescribed processing of body temperature data obtained from measurement with thermometer probe 46. When requested, the body temperature data is output to display unit 18 via cable 47. CPU 15 of display unit 18 inputs the body temperature data from thermometer main body 45 via display-side I/F 14.

Data for identifying the subject is previously attached to the body temperature data input by CPU 15. CPU 15 inputs the body temperature data and stores in association with the blood pressure measurement data which is previously measured and stored for each subject in display memory 12. The body temperature data and the blood pressure measurement data may be stored for each measurement time. Contents of display memory 12 additionally storing the body temperature data can be displayed on display portion 13 by an operation of display operation unit 16.

Though display unit 18 and thermometer main body 45 are connected to each other via cable 47 in FIG. 6, they may be connected with radio communication such as infrared communication.

In addition, thermometer main body 45 can access to the contents of display memory 12 via cable 47. More specifically, when the subject operates an operation unit 48 of thermometer main body 45 to instruct to display the measurement data on a side of display unit 18, the microcomputer of thermometer main body 45 requests the measurement data of CPU 15 of display unit 18 based on this instruction. CPU 15 reads the measurement data from display memory 12 responsive to the request and outputs the data to a side of thermometer main body 45 via display-side I/F 14 and cable 47. The microcomputer of thermometer main body 45 inputs the measurement data output from display unit 18, performs prescribed processing and displays the result on a display unit 49.

In addition, thermometer main body 45 may be connected via cable 47 to blood pressure measurement device 50 having display unit 18 mounted thereon, as shown in FIG. 5A. As an interface in blood pressure measurement device 50 for connecting cable 47 in this situation, a separate interface other than main body-side I/F 9 or a port of main body-side I/F 9 other than that for display-side I/F 14, which is not shown, can be used. CPU 10 of main body unit 51 processes an instruction or data from thermometer main body 45 input from the separate interface or the port, and updates (additionally stores the body temperature data) or reads contents of process memory 17 or display memory 12 using the result of processing. Data read is transmitted to thermometer main body 45 via main body-side I/F 9 and cable 47 and displayed on display unit 49.

Though the body temperature data is stored in addition to the blood pressure measurement data in display memory 12 with thermometer main body 45 which is separate from blood pressure measurement device 50, the data in display memory 12 may be deleted. After the subject ensures the display on display unit 49, for example, the subject operates operation unit 48 to give an instruction to delete desired data of displayed data, which instruction is provided to CPU 15 of display unit 18. CPU 15 then deletes the desired data from display memory 12 based on the instruction. The instruction for deleting may be given by an operation of display operation unit 16 or main body operation unit 11.

Although the present invention has been described and illustrated in detail, it is clearly understood that the same is by way of illustration and example only and is not to be taken by way of limitation, the spirit and scope of the present invention being limited only by the terms of the appended claims.

1-6. (canceled)

7. A blood pressure measurement device, comprising:
a main body unit fixed to said blood pressure measurement device and a display unit removably mounted on said blood pressure measurement device; wherein
said main body unit includes
a cuff mounted on a region for measurement of a living body and a main body operation unit operated to externally provide an instruction for a blood pressure measurement operation,
said cuff is integrally or separately attached to said main body unit, and
said display unit includes
a storage unit for storing data of blood pressure measurement, a display portion for displaying measurement data in said storage unit, and a display operation unit operated to externally provide an instruction for a display operation of said display portion.

8. A blood pressure measurement device, comprising:
a main body unit fixed to said blood pressure measurement device and a display unit removable mounted on said blood pressure measurement device; wherein
said main body unit includes
a cuff mounted on a region for measurement of a living body, and a control unit for measuring a blood pressure of said living body with adjusting a pressure of said cuff for blood pressure measurement,
said cuff is integrally or separately attached to said main body unit, and
said display unit includes
a storage unit for storing data of blood pressure measurement with said control unit, a display portion for displaying measurement data in said storage unit, a main body operation unit operated to externally provide an instruction for a blood pressure measurement operation of said control unit and a display operation unit operated to externally provide an instruction for a display operation of said display portion.
